(19) United States
(12) Patent Application Publication
(10) Pub. No.: US 2024/0290498 A1
(43) Pub. Date: Aug. 29, 2024

(54) SYSTEM AND METHOD FOR DETERMINING BLOOD PRESSURE FROM ELECTRICAL IMPEDANCE MEASUREMENTS

(71) Applicant: University of Utah Research Foundation, Salt Lake City, UT (US)

(72) Inventor: Benjamin Sanchez Terrones, Salt Lake City, UT (US)

(21) Appl. No.: 18/586,197

(22) Filed: Feb. 23, 2024

Related U.S. Application Data
(60) Provisional application No. 63/455,114, filed on Mar. 28, 2023.
(60) Provisional application No. 63/448,180, filed on Feb. 24, 2023.

(51) Int. Cl. G16H 50/30; G06F 17/13; G16H 40/60

(52) U.S. Cl. CPC G16H 50/30; G06F 17/13; G16H 40/60

(57) ABSTRACT

Disclosed are a system and method for predicting blood pressure from electrical impedance measurements at the skin. The system includes a wearable device that is configured to be worn against the skin (or attached directly to the skin) above a target vessel, such as a target artery. The wearable device includes an array of electrodes that enable measurement of electrical impedance at the skin. The system receives a series of electrical impedance measurements from the wearable device, and based on the series of electrical impedance measurements, calculates a blood pressure prediction using a predictive model that is informed by the incompressible Navier-Stokes equations for a tube with elastic boundary conditions and by the Maxwell-Fricke equations relating red blood cell orientation and distribution to blood conductivity.

### CROSS-REFERENCE TO RELATED APPLICATIONS

This application claims priority to and the benefit of U.S. Provisional Patent Application No. 63/448,180, filed Feb. 24, 2023 and to U.S. Provisional Patent Application No. 63/455,114, filed Mar. 28, 2023, each of which is incorporated herein in its entirety by reference.

### BACKGROUND

### Technical Field

This disclosure relates to a system and method for determining blood pressure by measuring skin impedance above a target artery.

### Related Technology

Measurement of blood pressure (BP) is essential for early diagnosis and management of hypertension, a condition that 45% of US adults have and a risk factor for development of heart failure, the leading cause of death in the US. Compared to ambulatory BP measurements, frequent out-of-clinic BP measurements are better predictors of cardiovascular events but, unfortunately, existing technologies requires costly and cumbersome instrumentation setups that prevent their use outside of the clinic or the lab. Accurate out-of-clinic BP measurements still require the use of automated inflation cuffs; however, the inflation of the cuff during measurement is known to trigger an increase in BP, and ambulatory BP measurements during the night cause sleep arousal and stress, leading to higher BP readings. Further, regular adult size cuffs do not fit all subjects, which can result in misleading BP readouts.

Some approaches have removed the need for an inflation cuff by relying on pulse transit time (PTT) or Pulse Arrival Time (PAT) measurements, which are related to pulse wave velocity (PWV) and BP through Moens-Korteweg and Hughes equations. These methods combine whole-body wrist-to-ankle impedance cardiography (ICG) and distal impedance plethysmogram channel (IPG) measuring changes between the knee joint level and the calf, with three-lead ECG. Then, when the pulse pressure wave enters the aortic arch and the diameter of the aorta changes, the whole-body impedance decreases. By measuring the time difference between the onset of the decrease in impedance in the whole-body ICG signal and, later, the popliteal artery signal as detected by the distal IPG, the PWV can be determined knowing the distance L and the time difference between the two recording sites. Alternatively, PTT can also be determined by measuring ECG and PPG waveforms, or two PPG waveforms and detecting again the time delay between these waveforms. These approaches are complex and inconvenient, however, and have limited use outside the research setting for continuous BP monitoring.

Another approach has proposed the use of impedance-detected time-delays recorded over a relatively short distance to infer BP. See Bassem Ibrahim and Roozbeh Jafari, Cuffless blood pressure monitoring from a wristband with calibration-free algorithms for sensing location based on bio-impedance sensor array and autoencoder, Scientific Reports, 12(1):319, December 2022. Their approach used 18 electrodes placed in the wrist to extract “whole body PTT-like” time-delay differences between different combinations of electrodes, and then fed these data into a black-box machine learning model to infer systolic and diastolic BP. In particular, these authors used a convolutional neural network (CNN) autoencoder to reduce the dimension of the impedance input signal and then estimated the arterial pulsation in the latent space making a regression to predict systolic/diastolic BP. This approach is not a real-time algorithm, but rather feeds the entire impedance signal at once into the CNN to provide offline values. Moreover, this approach does not result in a predictive model rooted in physics and is therefore unable to generate physiologically explainable BP predictions.

Accordingly, there is an ongoing need for systems and methods that can continuously determine BP in a more convenient manner, without requiring conventional inflation cuff devices, and without relying on “black box” machine learning algorithms untethered to real physiology.

### SUMMARY

This disclosure relates to a system and method for predicting blood pressure from electrical impedance measurements at the skin. The system includes a wearable device that is configured to be worn against the skin (or attached directly to the skin) above a target blood vessel, such as a target artery. The wearable device includes an array of electrodes that enable measurement of electrical impedance at the skin. The system receives a series of electrical impedance measurements from the wearable device, and based on the series of electrical impedance measurements, calculates a blood pressure prediction using a predictive model that is informed by the incompressible Navier-Stokes equations for a tube with elastic boundary conditions and by the Maxwell-Fricke equations relating red blood cell orientation and distribution to blood conductivity.

Blood pressure can be related to the shear stress (and thus red blood cell orientation and deformation) using the Navier-Stokes equations for an elastic tube. The red blood cell orientation and distribution can be related to the blood conductivity using the Maxwell-Fricke equations. Electrostatic principles describe the relationship between blood conductivity and the electrical impedance measured at the skin. Accordingly, while a forward analytical model can start with blood pressure to predict electrical impedance measured at the skin, predicting blood pressure from measured electrical impedance represents a computational inverse problem.

The system and method described herein approach this inverse problem using machine learning algorithms that are beneficially informed by physiologically relevant fluid dynamic and electrical principles, thereby providing physiologically meaningful blood pressure predictions. In contrast, without involving proper physics in the training loop, physics-free machine learning methods are less able to generalize to “unseen” impedance data and have reduced predictive accuracy for blood pressure, particularly in extreme cases such as hypotensive and hypertensive conditions.

In one embodiment, the predictive model includes a physics-informed neural network (PINN), the PINN being trained to minimize a first loss function, wherein the first loss function incorporates the incompressible Navier-Stokes equations for a tube with elastic boundary conditions. The predictive model can further include a decoder neural network configured to output an electrical impedance prediction based on the blood pressure prediction, the decoder neural network being trained to minimize a second loss function that determines loss between measured impedance and the impedance prediction, and the PINN being further trained to minimize the second loss function.

The decoder neural network can be trained using a training dataset that uses a forward model relating blood pressure to electrical impedance measured at the skin. The forward model incorporates (1) a physiological model that uses (i) Navier-Stokes equations relating blood pressure to shear stress and red blood cell orientation and (ii) Maxwell-Fricke equations relating red blood cell orientation to blood conductivity, and (2) an electrostatic model relating blood conductivity to impedance measured at the skin.

The electrostatic model can include a muscle tissue layer, a subcutaneous adipose tissue (SAT) layer, and a dermal tissue layer, with a target vessel region embedded within the muscle tissue layer. The predictive model can further include a preliminary encoder neural network that functions to reduce dimensionality of a resistance signal before passing a reduced-dimension resistance signal to the PINN. This gives the PINN the form of a “signal-tagged” PINN (SPINN).

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

The patent or application file contains at least one drawing executed in color. Copies of this patent or application publication with color drawing(s) will be provided by the Office upon request and payment of the necessary fee.

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an overview of a system for predicting blood pressure from electrical impedance measured at the skin;

FIG. 2 is a flow chart providing an overview of a method for predicting blood pressure from electrical impedance measured at the skin;

FIGS. 3A and 3B illustrate example architectures of a predictive model for predicting blood pressure from measured electrical impedance while considering the governing fluid dynamic and electrical principles underlying the relationship;

FIG. 4 is an example list of parameter values for a forward model that relates blood pressure to impedance measured at the skin using physiological and electrostatic principles, and which is usable to inform the predictive machine learning model;

FIG. 5 shows an electrostatic model that includes multiple tissue layers with an embedded blood vessel;

FIG. 6 illustrates an example method of generating a synthetic training dataset of paired blood pressure and impedance measurements from real blood pressure measurements;

FIG. 7A is a graph showing results of testing the electrostatic model and indicating a resistance relationship with the radial artery radius and blood conductivity;

FIG. 7B shows results of a variance-based global sensitivity analysis determining that BP was expected to have the largest influence on the time of the maximum and minimum of the resistance signal;

FIG. 8A shows major anatomical features of the left wrist used in finite-element method electrostatic simulations of left wrist impedance measurements using the electrostatic model;

FIGS. 8B-8H illustrate results of parameter sweeps for blood conductivity, electrode position, radial artery depth, and radial artery radius using the electrostatic model;

FIGS. 81 and 8J illustrate a volume impedance density (VID) analysis at 50 kHz using the electrostatic model;

FIG. 9A shows the results of using a combined fluid dynamic and electrostatic model (full forward model) to relate brachial blood pressure waveforms all the way to impedance measured at the wrist;

FIG. 9B shows results of a variance-based global sensitivity analysis determining that blood pressure is a significant contributor to aspects of the impedance signal;

FIG. 9C compares the relative impacts of red blood cell (RBC) orientation and RBC deformation within the forward model; and

FIGS. 10A-10C are three sample blood pressure waveforms (BPᵢ) with their corresponding resistance (Rᵢ) and predicted blood pressure ( [symbol] ) predicted using the predictive model, showing that the predictive model was able to generate predicted blood pressure that effectively corresponds to the reference blood pressure.

### DETAILED DESCRIPTION

### Overview

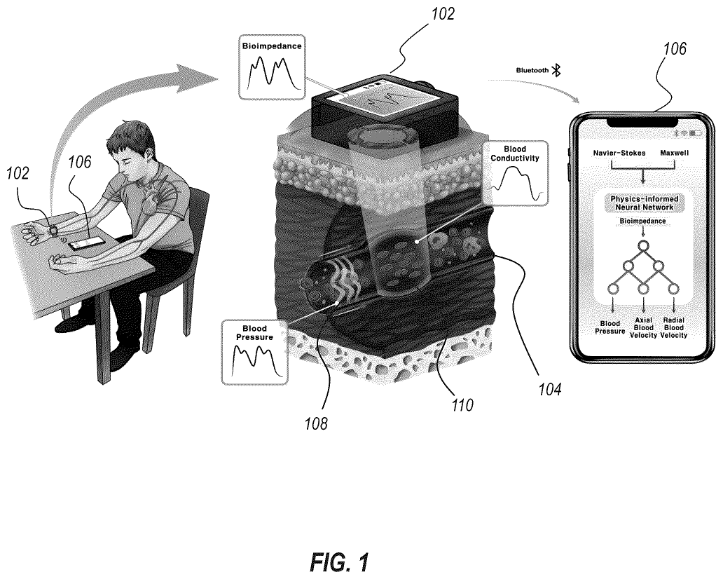

FIG. 1 illustrates an overview of a system 100 for predicting blood pressure from electrical impedance measurements in real time. The system includes a wearable device 102 that is configured to be worn against the skin (or attached directly to the skin) above a target vessel, such as a target artery. The wearable device 102 includes an array of electrodes (e.g., current-injecting and voltage-sensing) that enable measurement of electrical impedance at the skin. In the illustrated example, the wearable device 102 has a wristwatch form factor. In other embodiments, the wearable device 102 can have other form factors, such as a patch form factor, a leg wrap form factor, or any other suitable form factor that enables placement of the electrode array against the skin above a target vessel.

The target vessel can also be varied according to particular application needs. For example, while FIG. 1 shows a wearable device 102 configured for placement above the radial artery 104 of the wrist, other embodiments can configure the wearable device 102 for placement over other target vessels, including carotid arteries, brachial arteries, femoral arteries, popliteal arteries, dorsalis pedis arteries, internal pudendal artery, and dorsal artery of the penis, for example. The target vessel can also be a vein, such as the cephalic vein, basilic vein, median cubital vein, great saphenous vein, and small saphenous vein, for example.

While most of the examples described herein use a wristwatch-like wearable device 102 situated above the radial artery 104, it will be understood that the same components and principles can be applied to other form factors and/or other target vessels to predict blood pressure.

The system 100 includes one or more processors and one or more hardware storage devices that enable the system 100 to carry out the functions that enable predicting blood pressure. The processing functions can be carried out on the wearable device 102 and/or on one or more external devices 106, such as a mobile device and/or desktop computer device. Presently preferred embodiments communicate with one or more external devices 106 to offload more processing from the wearable device 102 and to leverage the user interface of the external device(s) 106.

In the illustrated embodiment, the wearable device 102 includes a communications module that enables communication with one or more external devices 106 to allow for transfer of data (e.g., the measured electrical impedance), instructions, requests, or other queries. The communication can be via a wireless connection, such as a radio frequency (RF) wireless connection (e.g., Bluetooth®). Alternative embodiments can additionally or alternatively enable wired connections between the wearable device 102 and the one or more external devices 106.

As illustrated in FIG. 1, a propagating blood pressure wave 108 causes the elastic walls of the vessel to expand while the local shear rate also moves the coincident red blood cells (RBCs) 110 into a more ordered orientation. This shift in red blood cell orientation, as well as the simultaneous deformation red blood cell shape, contributes to changes in blood conductivity. The change in blood conductivity in turn changes the electrical impedance measured at the skin above the vessel.

Blood pressure can be related to the shear stress (and thus red blood cell orientation and deformation) using the Navier-Stokes equations for an elastic tube. The red blood cell orientation and distribution can be related to the blood conductivity using the Maxwell-Fricke equations. Electrostatic principles describe the relationship between blood conductivity and the electrical impedance measured at the skin. Accordingly, while a forward analytical model can start with blood pressure to predict electrical impedance measured at the skin, predicting blood pressure from measured electrical impedance represents a computational inverse problem.

As described in more detail below, the system and method described herein approach this inverse problem using machine learning algorithms that are beneficially informed by physiologically relevant fluid dynamic and electrical principles, thereby providing physiologically meaningful blood pressure predictions. In contrast, without involving proper physics in the training loop, physics-free machine learning methods are less able to generalize to “unseen” impedance data and have reduced predictive accuracy for blood pressure, particularly in extreme cases such as hypotensive and hypertensive conditions.

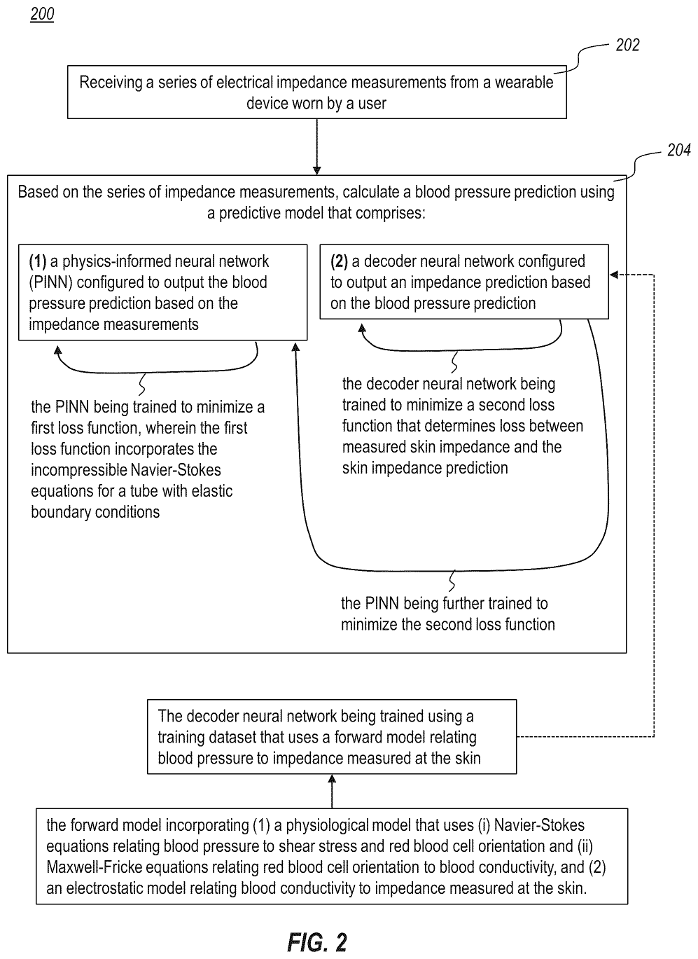

FIG. 2 is a flow chart providing an overview of a method 200 for predicting blood pressure from electrical impedance measured at the skin. The method 200 can be carried out using the system 100, for example. The method 200 includes receiving a series of electrical impedance measurements from a wearable device worn by a user (step 202). Based on the series of impedance measurements, the system calculates a blood pressure prediction using a predictive model (step 204). The predictive model can comprise a physics-informed neural network (PINN) configured to output the blood pressure prediction based on the impedance measurements. The PINN can be trained to minimize a first loss function, wherein the first loss function incorporates the incompressible Navier-Stokes equations for a tube with elastic boundary conditions.

The predictive model can further comprise a decoder neural network configured to output an impedance prediction based on the blood pressure prediction. The decoder neural network can be trained to minimize a second loss function that determines loss between measured skin impedance and the skin impedance prediction. The decoder neural network can be leveraged to further train the PINN to minimize the second loss function. Accordingly, the PINN and the decoder neural network can be trained separately and in tandem.

The decoder neural network can be trained using a training dataset that uses a forward model relating blood pressure to electrical impedance measured at the skin. As explained in more detail below, the forward model can incorporate (1) a physiological model that uses (i) Navier-Stokes equations relating blood pressure to shear stress and RBC orientation/deformation and (ii) Maxwell-Fricke equations relating red blood cell orientation/deformation to blood conductivity, and (2) an electrostatic model relating blood conductivity to impedance.

The predictive model can further comprise a preliminary encoder neural network that functions to reduce dimensionality of a resistance signal before passing a reduced-dimension resistance signal to the PINN. A PINN that is adapted in this manner is referred to herein as a “signal-tagged” PINN (SPINN).

The neural networks of the predictive model can independently take the form of any suitable neural network, such as CNNs. In one embodiment, the preliminary encoder neural network is a 1-dimensional CNN (1D-CNN) and the decoder neural network is a 2-dimensional CNN (2D-CNN).

The blood pressure prediction can beneficially be determined in real time. This contrasts with certain prior methods such as the Jafari method discussed above. That is, rather than feeding an entire impedance signal to the model at once, the disclosed predictive model can instead output a current blood pressure prediction. As used herein, “real time” means that the output blood pressure prediction is temporally linked to the occurrence of the corresponding impedance measurement with minimal lag (e.g., no more than 1 minute, no more than 30 seconds, no more than 10 seconds, no more than a few seconds) such that obtained blood pressure prediction corresponds to the user's actual current blood pressure.

The system 100 can beneficially provide continuous and accurate blood pressure determinations for a user. This can further comprise helpful notifications and/or care recommendations. For example, the system can generate notifications indicating blood pressure trends, indicating the occurrence of concerning or dangerous blood pressure levels, or providing recommendations to see a healthcare professional, for example. The blood pressure determinations can beneficially provide the user and/or healthcare professionals with effective blood pressure data and trends that can enable more-informed healthcare decisions and improved outcomes.

Training data for the predictive model can comprise real blood pressure measurements with simultaneous impedance measurements of patients/subjects. Training data can additionally or alternatively include synthetic training datasets. For example, publicly available blood pressure databases can be leveraged to generate corresponding impedance signals by passing the blood pressure data through the forward model. While such synthetic datasets lack the correspondence of datasets generated from real patients, they have the advantage of being relatively large in sample number due to the relatively large size of the available blood pressure databases.

The predictive model can be configured to update as additional user data is gathered. For example, user data (e.g., anonymized) from a plurality of users can be gathered from respective wearable devices 102 and/or external devices 106 into a central database from which model updates can be derived as additional user data is gathered.

### Example Predictive Model

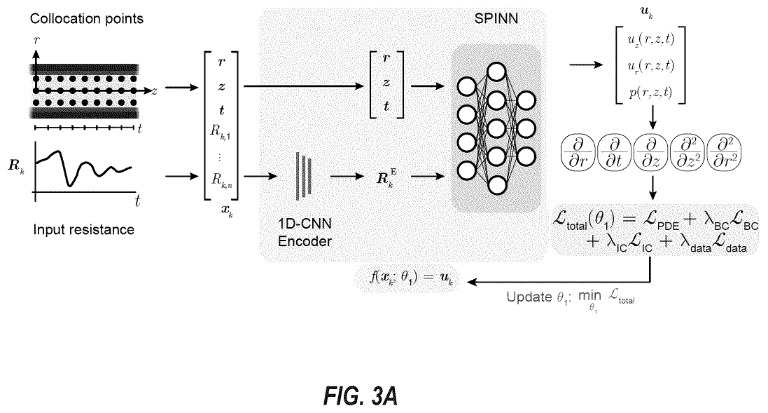

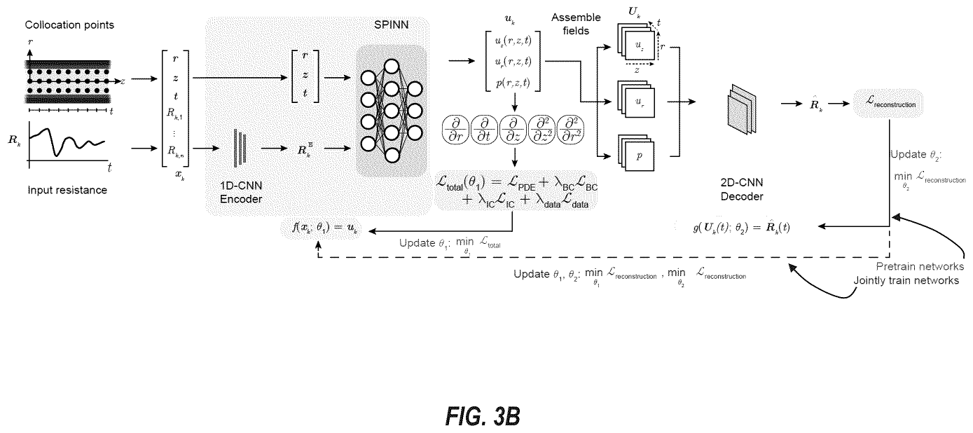

An example predictive model, usable in system 100 and method 200, is shown in greater detail in FIGS. 3A and 3B. The illustrated architectures beneficially predict blood pressure from measured electrical impedance while considering the governing fluid dynamic and electrical principles underlying the relationship.

FIG. 3A illustrates the first stage of the predictive model, which receives the input resistance signal and outputs blood pressure and blood velocity (radial and axial) vectors. The associated loss function (“the first loss function”) has three goals: (i) the predicted blood pressure should agree with data, (ii) the blood pressure and velocities should satisfy the biophysical partial-differential equations (Navier-Stokes), and (iii) boundary conditions at the vessel wall should be satisfied.

In more detail, the first stage maps a space-time vector (r, z, t) and a resistance signal Rₖ to pressure and axial-radial velocity fields [p, u_{z}, uᵣ]k (r, z, t), evaluated at (r, z, t). The resistance signal, Rₖ, first passes through a 1D-CNN encoder and the reduced-dimensional representation, Rₖ^{E}, along with (r, z, t) is passed to a signal-tagged physics informed neural network (SPINN) model, which functions as a PINN and further augments the space-time input with an additional (encoded) resistance signal, Rₖ^{E}. The model can make interpretable, space-time bloodflow field predictions that (i) are dependent on the data sample k and (ii) are grounded in physics; in particular, the model pushes the fields to satisfy pulsatile fluid flow in an elastic tube, described by the incompressible Navier-Stokes equations in a cylindrical tube with elastic boundary conditions.

The loss function of the first stage can take the form:

$${L}_{t o t} : = {L}_{p d e} + {λ}_{i c} {ℒ}_{i c} + {λ}_{bc} {ℒ}_{bc} + {λ}_{data} {ℒ}_{data}$$

where λ_{ic}, λ_{bc}, and λ_{data} are model weights, and

$${ℒ}_{pde} := {\sum }_{i ∈ [ {N}_{pde} ]} {\sum }_{j \leq 3} {e}_{j}^{2} ( {u}_{z , i} , {u}_{r , i} , {p}_{i} , {r}_{i} )$$

$${ℒ}_{ic} := {\sum }_{i ∈ [ {N}_{ic} ]} {[ p ( {r}_{i} , {z}_{i} , {t}_{i} = 0 ) - {p}^{ℱ} ( {r}_{i} , {z}_{i} , {t}_{i} = 0 ) ]}^{2} + {[ {u}_{z} ( {r}_{i} , {z}_{i} , {t}_{i} = 0 ) - {u}_{z}^{ℱ} ( {r}_{i} , {z}_{i} , {t}_{i} = 0 ) ]}^{2}$$

$${ℒ}_{bc} := {\sum }_{i ∈ [ {N}_{bc} ]} {u}_{z}^{2} ( {r}_{i} , {z}_{i} , {t}_{i} ) + {[ {u}_{r} ( {r}_{i} , {z}_{i} , {t}_{i} ) - {\partial }_{t} η ( {z}_{i} , {t}_{i} ) ]}^{2}$$

$${ℒ}_{data} := {\sum }_{i ∈ [ {N}_{data} ]} {[ p ( {r}_{i} , {z}_{i} , {t}_{i} ) - {p}^{ℱ} ( {r}_{i} , {z}_{i} , {t}_{i} ) ]}^{2} + {[ {u}_{z} ( {r}_{i} , {z}_{i} , {t}_{i} ) - {u}_{z}^{ℱ} ( {r}_{i} , {z}_{i} , {t}_{i} ) ]}^{2}$$

$${ℒ}_{pde} {e}_{j} ( {u}_{z , i} , {u}_{r , i} , {p}_{i} , {r}_{i} ) {N}_{pde} {ℒ}_{ic} {N}_{ic} {t}_{i} = 0 {ℒ}_{bc} {N}_{bc} {\partial }_{t} η ( z , t ) = {u}_{r} ( r = \hat{a} + η ( z , t ) , z , t ) {u}_{r} \hat{a} {ℒ}_{data} {N}_{data} {z}_{i} ∈ \{ 0 , l \} {p}^{ℱ} ( {r}_{i} , z = 0 , {t}_{i} )$$

is a squared sum of Navier-Stokes residuals evaluated at a batch of collocation points; is a squared sum of the discrepancy between the predicted and prescribed initial conditions, evaluated at a batch of collocation points with; is a squared sum of the discrepancy between the predicted and prescribed (no-slip, no-penetration) boundary conditions, evaluated at a batch of collocation points—the no-penetration condition is, where η is the arterial wall displacement, is the radial velocity and is the rest arterial radius; and is a squared sum of the discrepancy between the predicted and prescribed inlet and outlet boundary conditions, evaluated at a batch of collocation points, where. The term

$${ℒ}_{pde} {e}_{j} ( {u}_{z , i} , {u}_{r , i} , {p}_{i} , {r}_{i} ) {N}_{pde} {ℒ}_{ic} {N}_{ic} {t}_{i} = 0 {ℒ}_{bc} {N}_{bc} {\partial }_{t} η ( z , t ) = {u}_{r} ( r = \hat{a} + η ( z , t ) , z , t ) {u}_{r} \hat{a} {ℒ}_{data} {N}_{data} {z}_{i} ∈ \{ 0 , l \} {p}^{ℱ} ( {r}_{i} , z = 0 , {t}_{i} )$$

represents real data.

The predictive model can optionally further comprise a second stage to further inform the model according to the underlying physiological and electrostatic principles. FIG. 3B illustrates the combined first and second stages of the predictive model. The second stage of the model uses a 2D-CNN to map the pressure and velocity fields [p, u_{z}, uᵣ] (rᵢ, zⱼ, t), evaluated at a grid of (rᵢ, zᵢ) points and time t to a resistance signal R(t), evaluated at time t. This stage can be viewed as a forward model for predicting the impedance from the fluid flow.

Together, the two stages function as a physics-informed autoencoder. A resistance signal and space-time vector are encoded in the first stage (1D-CNN+SPINN) to give pressure velocity fields at that space-time. These fields are decoded in the second stage (2D-CNN) to recover the resistance signal. This architecture solves both a forward problem and inverse/prediction problem and beneficially allows several different types of model loss to be evaluated (physics, predictive error, autoencoder reconstruction).

The loss function of the second stage (“the second loss function”) can take the form:

$${ℒ}_{decode} = {\sum }_{k ∈ [ K ]} {\sum }_{i ∈ [ n ]} {[ {R}_{k} ( {t}_{i} ) - {\hat{R}}_{k} ( {t}_{i} ) ]}^{2}$$

where Rₖ (tᵢ) is the predicted resistance signal at tᵢ, i∈[n] and \hat{R}ₓ(tᵢ) is the k-th reference resistance signal, at tᵢ, i∈[n].

Training of the predictive model can include pre-training each stage separately and then in tandem. Training the first stage can include random sampling of a batch of collocation points and passing the points together with a resistance signal though the ID-CNN+SPINN model to predict the vessel fluid fields. These are used to evaluate the first loss function and derivatives with respect to model parameters and to update the model parameters. Training the second stage can include evaluating the pressure and velocity (axial and radial) fields [p, u_{z}, uᵣ], at a grid of (rᵢ, zⱼ) points and time t using the forward analytical model described herein. These channels can be passed through the 2D-CNN to predict resistance, and then used to evaluate the second loss function and update the model parameters.

To train the stages in tandem, the first stage evaluates the pressure and velocity fields [p, u_{z}, uᵣ], at a grid of (rᵢ, zⱼ) points and time t. These values are then reshaped and passed through the second stage to reconstruct the input resistance signal. The first stage model parameters can be alternatively updated according to both the [symbol] ₜₒₜ and [symbol] _{decode} loss functions.

### Example Forward Model Features

As discussed above, a forward model can be utilized to generate training data and to drive the predictive model to align output with physiological and electrostatic principles. The forward model incorporates (1) a physiological model that uses (i) Navier-Stokes equations relating blood pressure to shear stress and red blood cell orientation and (ii) Maxwell-Fricke equations relating red blood cell orientation to blood conductivity, and (2) an electrostatic model relating blood conductivity to impedance measured at the skin.

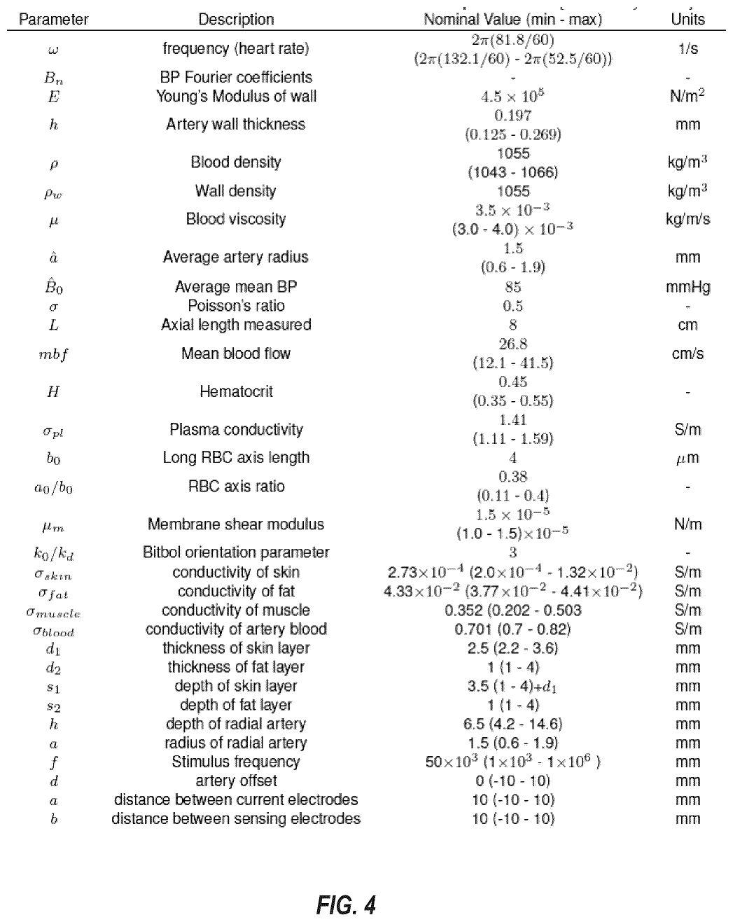

An example list of parameter values for the forward model is provided in the table illustrated in FIG. 4. These parameter values are examples only, and can be adjusted as needed for particular application needs and/or for particular patient/subject populations. For the physiological parameters (e.g., blood density, hematocrit, plasma conductivity), a normal distribution can be assumed.

### Physiological/Fluid Dynamic Model

Analytical solutions to reduced Navier-Stokes with an elastic tube boundary are used to model dynamics of the blood within the target vessel and potentially within connected or associated vessels. For example, in embodiments that use the radial artery as the target vessel, the forward model can be based on brachial artery blood pressure measurements with blood pressure waveform propagation modeled as branching between radial and ulnar arteries (e.g., in a Y-shaped geometry).

From the shear stresses within the target vessel, ellipsoidal RBCs are oriented and deformed with the flow, and Maxwell-Fricke theory can be used to compute a bulk conductivity measurement. The time-varying vessel radius and bulk conductivity computations can then be passed into the electrostatic model to compute electrical impedance at the skin.

Navier-Stokes can be first linearized according to the assumption that the radius of the target vessel is small compared to the wavelength of the propagating pressure wave. Solutions can be assumed to be time-periodic and space-varying with complex wave numbers. General solutions to these equations can be coupled with stress-strain equations of motion of a thin elastic tube wall. The model can assume an average artery radius of â, and a population average blood pressure of \hat{B}₀. This is done so that after a new blood pressure is inputted to the model with average pressure B₀, the vessel slightly “inflates” or “deflates” to match that pressure; this shift results in a resting vessel radius that can be defined by:

$$a = \hat{a} + \frac{( {B}_{0} - {\hat{B}}_{0} ) ( 1 - {σ}^{2} )}{h E} {\hat{a}}^{2}$$

where a is the resting arterial radius, σ is Poisson's ratio, h is the thickness of the arterial wall, and E is the Young's modulus of the wall. Coupling these equations allows explicit forms for the complex wave numbers, the pressure field, the pulsatile radial and axial fluid velocity fields, the arterial wall displacements, and the shear stress within the artery to be obtained.

For branched models such as those including the Y-shaped bifurcation from the brachial artery to the radial and ulnar arteries, these fluids solutions can provide a relationship between arterial pressure and volumetric flow rate such that assuming a Y-shaped bifurcation and assuming primary wave reflections at the branching point, a measured blood pressure waveform from upstream (e.g., the upper arm) can be propagated to a downstream point (e.g., the radial artery at the wrist). The linearized axial momentum Navier-Stokes equation can be integrated to obtain an equation describing a relationship between the rate of change of the flow rate, the pressure gradient, and the wall shear stress. Then, without relying on a Moens-Korteweg wave speed approximation, the explicit fluids solutions can be used to relate wall shear stress to flow rate, and the equation can be manipulated to derive admittance coefficients, defined as the ratio of the Fourier coefficients for flow rate and blood pressure, respectively. These admittance coefficients can then be used to compute reflectivity coefficients at the relevant branch point (e.g., the brachial-radial-ulnar branch point) to compute wave propagation dynamics through this point.

Shear stresses within the target vessel can then be fed into models in which ellipsoid red blood cells experience sheer stress-induced orientation and deformation, and Maxwell-Fricke theory can be used to tie this to bulk vessel conductivity measurements. Since the fluids solutions vary both radially and axially, the conductivity calculation can be measured not only over a cross-section of the target vessel but also down an axial segment of interest:

$${σ}_{blood} = \frac{2}{{a}^{2} L} {\int }_{0}^{L} {\int }_{0}^{a} r {σ}_{c v} ( r , z , t ) drdz$$

where σ_{blood} is the conductivity of the blood, L is the length of interest in the target vessel, and σ_{cv} is the conductivity of an infinitesimal control volume measured in the tube in radial coordinates, r, z, and t. The electrostatic model described herein can then be used to determine electrical impedance at the skin level from the changes in target vessel conductivity and radius over time.

Using a model that assumes elastic vessels rather than rigid vessels is beneficial. Rigid vessel models require knowledge of a pulsatile velocity time signal in order to construct a Fourier representation of the pressure gradient in the vessel. With an elastic model, only a blood pressure time signal is required to generate the solutions, making it easier to generalize to a population. Additionally, clastic tubes allow for the downstream propagation of pressure waves as solutions are not constant axially, allowing analysis of how an upstream pressure waveform attenuates down to the target location. A previous elastic artery model used a local flow theory in which small movements down the tube are assumed to not make large impacts on the velocity waveforms. This theory requires, however, additional assumptions to model the change in vessel radius with changes in pressure and knowledge of a center-line fluid velocity profile. In contrast, the disclosed model benefits in only requiring a blood pressure waveform to derive the full forms of the solutions.

For RBC modeling, ellipsoidal particles with short axis length a, and longer axes each with lengths b, are assumed to be distributed uniformly in the vessel with the blood stationary. The RBCs at this point are not deformed and are oriented randomly. Random orientation can mean that one of the axes is oriented axially with the tube, with each of the three axes equally likely to be oriented axially. As blood travels down the vessel, the RBCs orient (with one of their longer axes) in response to shear stress. In addition to orienting with the flow, the RBCs can deform or stretch, extending their long axes and shrinking their short axis, preserving volume, under shear stresses.

### Electrostatic Model

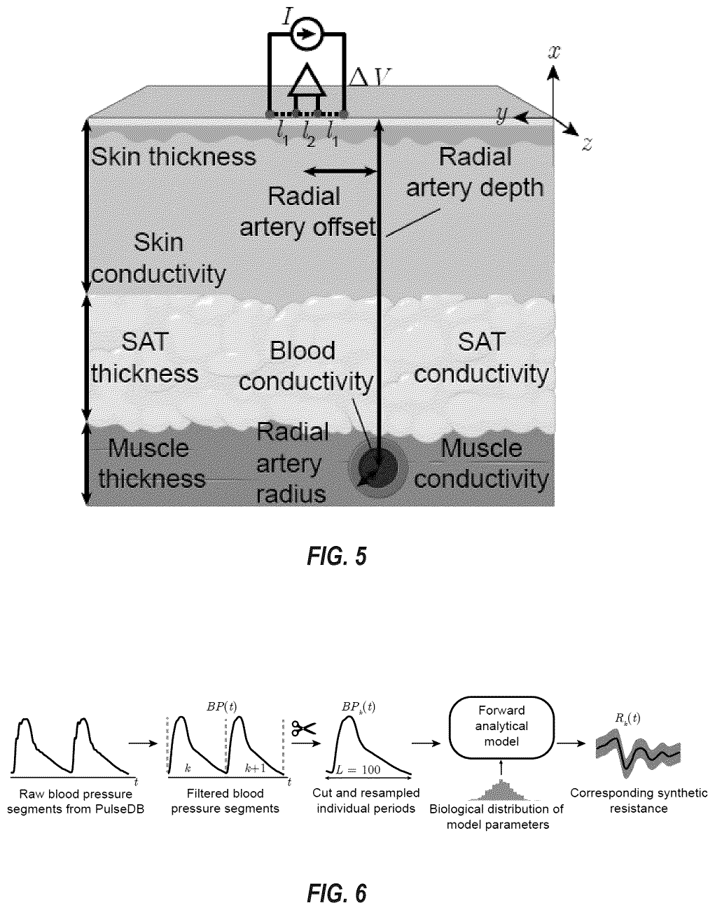

As shown in FIG. 5, the electrostatic model can include a multi-layered tissue model with embedded vessel. The model of FIG. 5 includes a muscle tissue layer, a subcutaneous adipose tissue (SAT) layer, and a dermal tissue layer, with a target vessel region embedded within the muscle tissue layer. The target vessel can be modeled as an infinitely long conductive cylinder embedded within the muscle layer. By solving Maxwell's equations under quasi-electrostatic approximation, the apparent electrical resistance arising from the superposition of a primary and secondary electric potential can be derived, where the primary potential ignores the vessel, and the secondary potential accounts for the potential contribution of the vessel. Approximating the reflection coefficient, RI, as

$$R 1 \approx - 1 + 2 ε and \frac{{σ}_{1}}{{σ}_{3}} \leq ε \leq \frac{{σ}_{1}}{{σ}_{2}}$$

yields closed form solutions for the primary and secondary potentials.

### Training Data Generated Using the Forward Model

As discussed above, training data for the predictive model can comprise real blood pressure measurements with simultaneous impedance measurements of patients/subjects and/or can comprise synthetic training datasets such as by passing publicly available blood pressure datasets through the forward model to generate corresponding impedance signals.

A synthetic training dataset can be improved by incorporating a biological distribution of one or more physiological parameters to relate blood pressure to impedance using the forward model. For example, certain physiological parameters relevant to the forward model include target vessel radius, mean blood flow, hematocrit, and/or plasma conductivity. These parameters can vary from subject to subject, and the population as a whole will exhibit a normal distribution for these values.

FIG. 6 illustrates an example method of generating such a synthetic training dataset. As shown, blood pressure waveform signals (e.g., from a public database) can be split into individual periods based on the maximum in the waveform. This ensures that there is no signal overlap. The blood pressure periods can then be passed through the forward model, in conjunction with a probability distribution of biological parameters to model biological variability, to synthetically generate the corresponding impedance periods.

### WORKING EXAMPLES

### 1. Electrostatic Model

An electrostatic model of the wrist was generated according to the framework shown in FIG. 5. This model was formally validated with finite element electrostatic simulations in COMSOL Multiphysics 5.5 (COMSOL Inc, Burlington, MA). The model layers included skin (2.5 mm), fat (1 mm), and muscle (infinite element domain). The simulations were performed in the electric currents module using a frequency domain study with Dirichlet boundary conditions for the current source (1 A) and ground surfaces defined as infinite element domains on the edges of the half space. The volume was adaptively meshed using free tetrahedrons with at least 2M voxels. The relative tolerance was set to 5e-4 and the maximum number of iterations were limited to 1e4. The maximum numerical error between the analytical and simulation models for an 8 mm region on the skin surface near the current source was <0.5%.

A global sensitivity analysis was performed to explore the model's sensitivity to parameters across their entire expected physiological range using a variance-based approach. We sampled blood pressure periods uniformly and used quasi-Monte Carlo sampling for the remaining parameter domain. For each sample drawn, we calculated the resulting mean resistance, peak-to-peak resistance, time of maximum resistance, and time of minimum resistance. We then assigned total order and first order Sobol indices for each parameter. The analysis considered over 1.8M data points in total. The infinite summations and integrals were individually analyzed and then truncated to ensure an error threshold less than 1e-3.

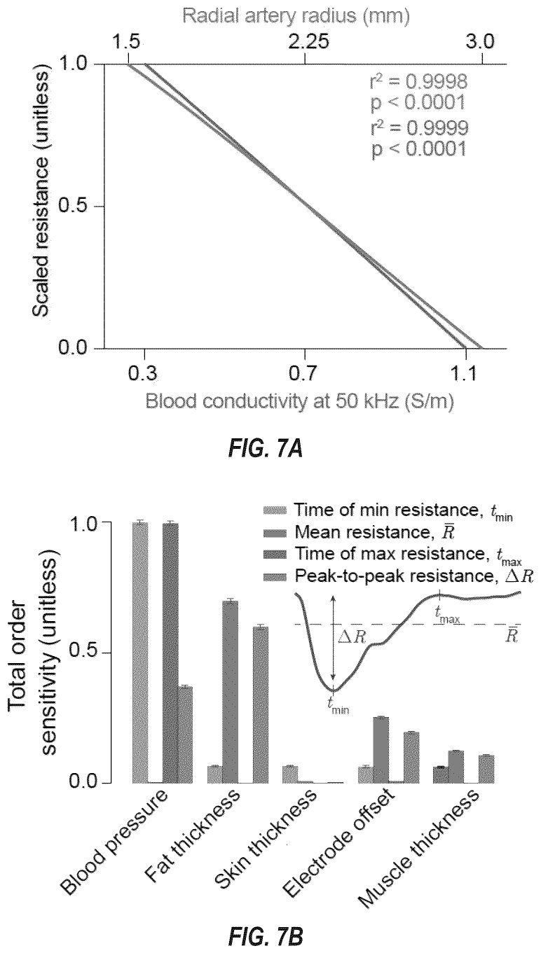

The model revealed an intimate resistance relationship with the radial artery radius (r2=0.9998, p<0.0001) and blood conductivity (r2=0.9999, p<0.0001) (FIG. 7A). The variance-based global sensitivity analysis determined that BP alone was expected to have the largest influence on the time of the maximum and minimum of the resistance signal with total order Sobol indices (mean+sd) of 0.999±0.004 and 0.998±0.005, respectively. The peak-to-peak resistance was found to be most influenced by subcutaneous adipose tissue (SAT) thickness with 0.532±0.005 and BP with 0.329±0.003. SAT thickness, electrode offset, and muscle thickness had the largest impact on the mean resistance with a contribution of 0.699±0.005, 0.253±0.002, and 0.123±0.001, respectively (FIG. 7B).

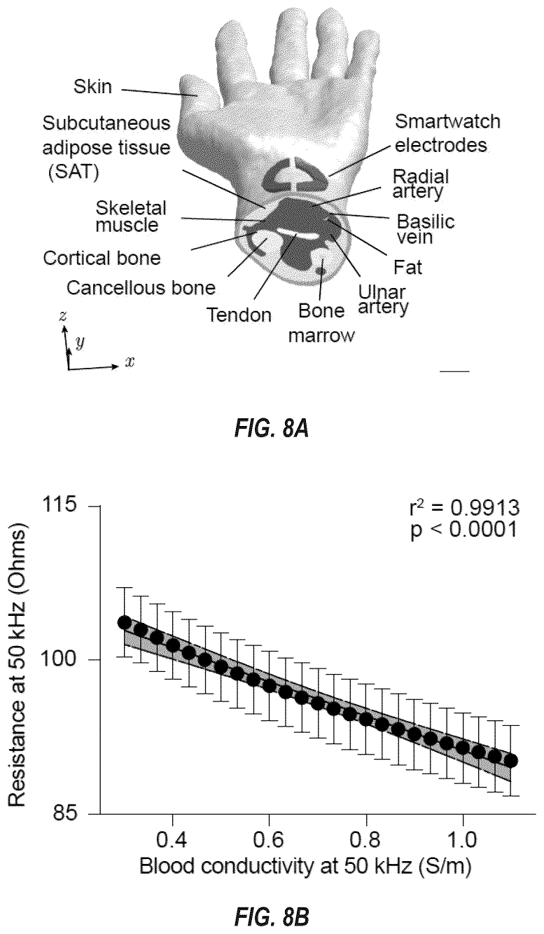

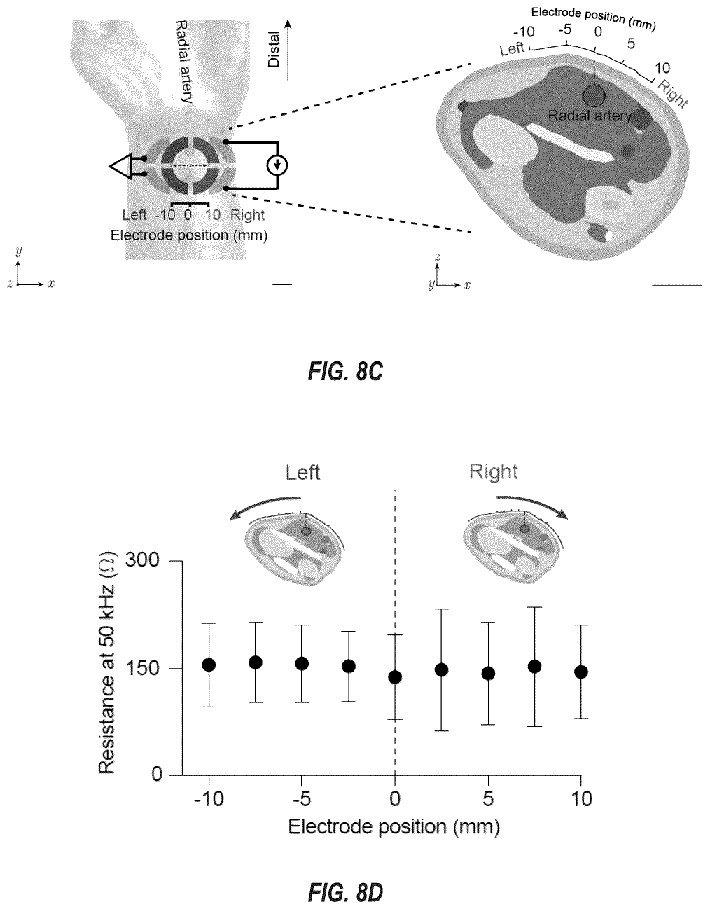

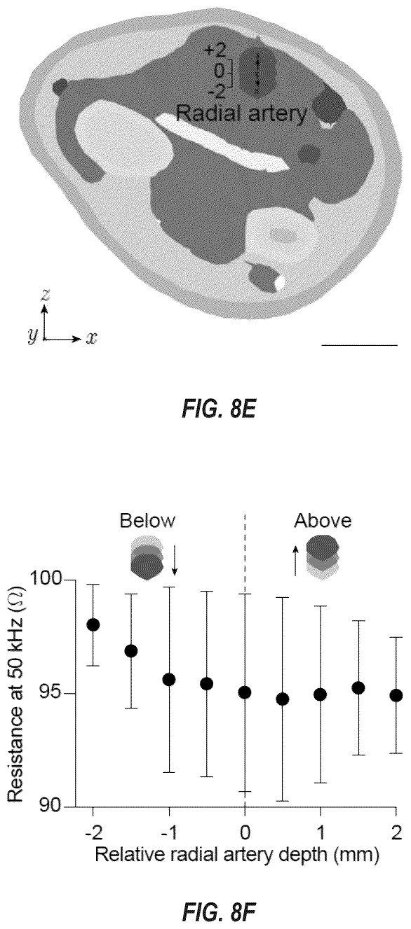

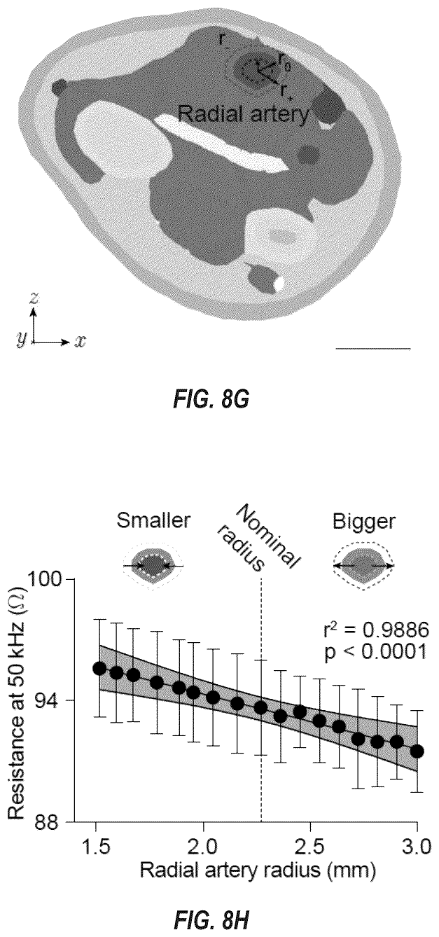

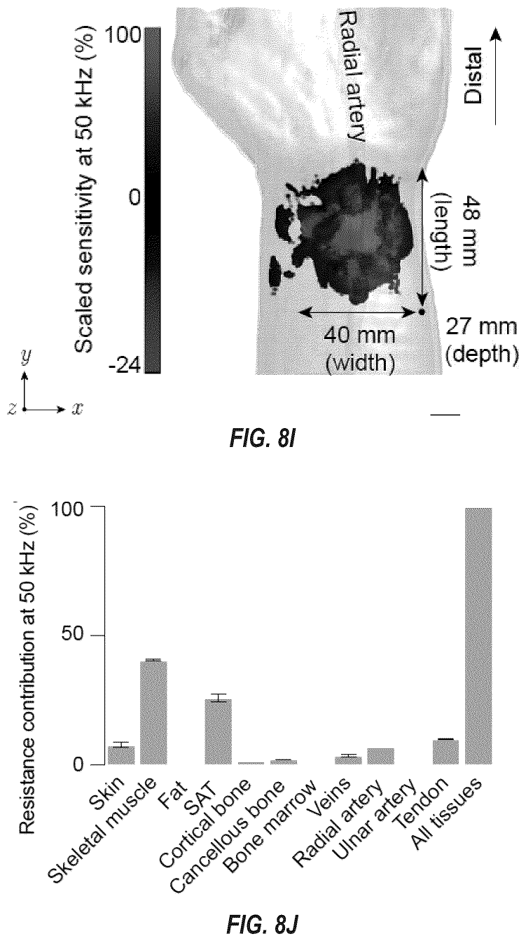

We also performed finite-element method electrostatic simulations of left wrist impedance measurements in Sim4Life V 7.2.1 (Zurich MedTech, Zurich Switzerland). FIG. 8A illustrates that the finite element models included the major anatomical features of the left wrist. We used the left forearms of the Ella, Morphed Ella, Fats, and Glenn version 3.1 human phantoms (age 43±27.7 years, body mass index 25±4.9 kg/m²). The smartwatch electrodes were modeled as perfect electric conductors on the left posterior wrist directly above the radial artery. The electrical properties of the tissues were assigned using the IT′IS database version 4.1 at the corresponding frequency. The simulation was solved using the electro-quasi-static module with 1 V Dirichlet boundary conditions, 1e-12 relative convergence tolerance, and a limit of 100,000 iterations.

We studied the impedance behavior of the left wrist by performing five parameter sweeps: frequency, blood conductivity, electrode position, radial artery depth, and radial artery radius. For the frequency sweep simulations, we included 25 logarithmically spaced frequencies between 1 kHz and 1 MHZ, the typical frequency range of impedance measurements.

For the conductivity sweep (FIG. 8B), we simulated 25 linearly-spaced blood conductivity values between 0.3 and 1.1 S/m. This range is centered around the nominal conductivity of blood at 50 kHz. We found blood's conductivity changes due to the cardiac cycle was intimately related with resistance changes at the wrist (r²=0.9913, p<0.0001).

For the electrode position sweep (FIGS. 8C and 8D), we moved the electrode configuration 10 mm left or right of the nominal position directly above the radial artery in 2.5 mm increments at 50 kHz. Moving the electrodes' location to account for subjects' different placement of the smartwatch resulted in a resistance change of 10.2%+14.2%. These differences were not significant, indicating that the system is robust to positioning of the wearable device within at least 10 mm to either side of the target blood vessel.

For the radial artery depth (FIGS. 8E and 8F), we moved the radial artery 2 mm above and below the nominal location in the human phantoms in 0.5 mm increments. Anatomic variation in the depth of the radial artery changed the resistance by 0.736%+1.608%. These differences were not significant, indicating that the system is robust to differing target artery depths within at least ±2 mm from a median target blood vessel depth.

To study the impact of the radial artery radius (FIGS. 8G and 8H), we incrementally inflated and deflated the radius at 17 values between 1.5 and 3.0 mm (i.e., the radial artery was incrementally stretched and shrunk to 65% and 135% of the nominal radius of 2.2 mm. Unless otherwise noted, tissues' electrical properties (conductivity and relative permittivity) were determined by the stimulus frequency and the electrode position was fixed at the nominal location directly above the radial artery. Results demonstrate a linear relationship with impedance (r²=0.9886, p<0.0001).

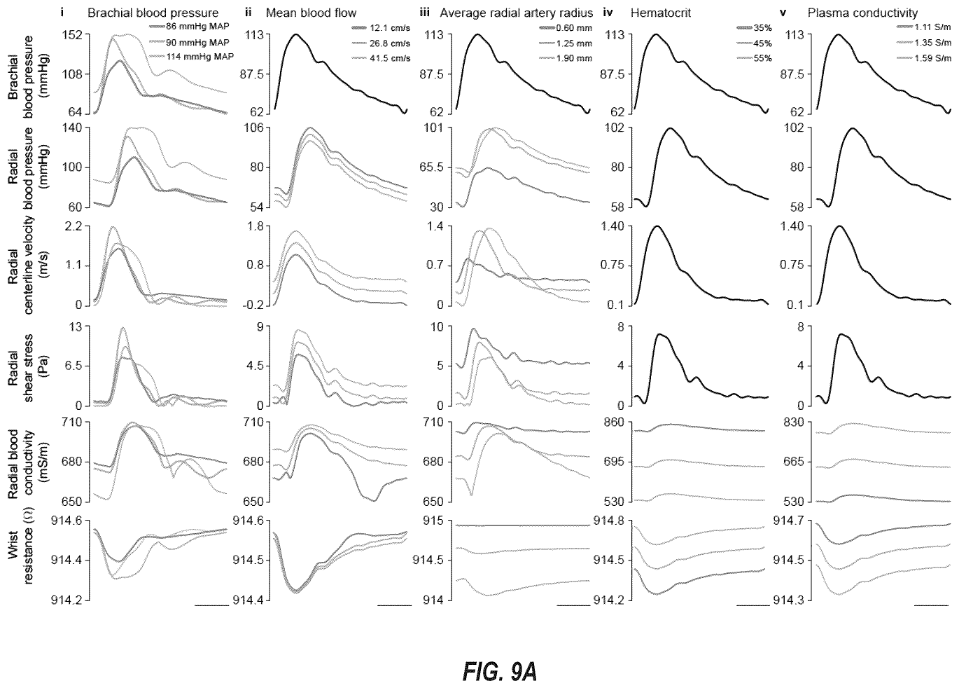

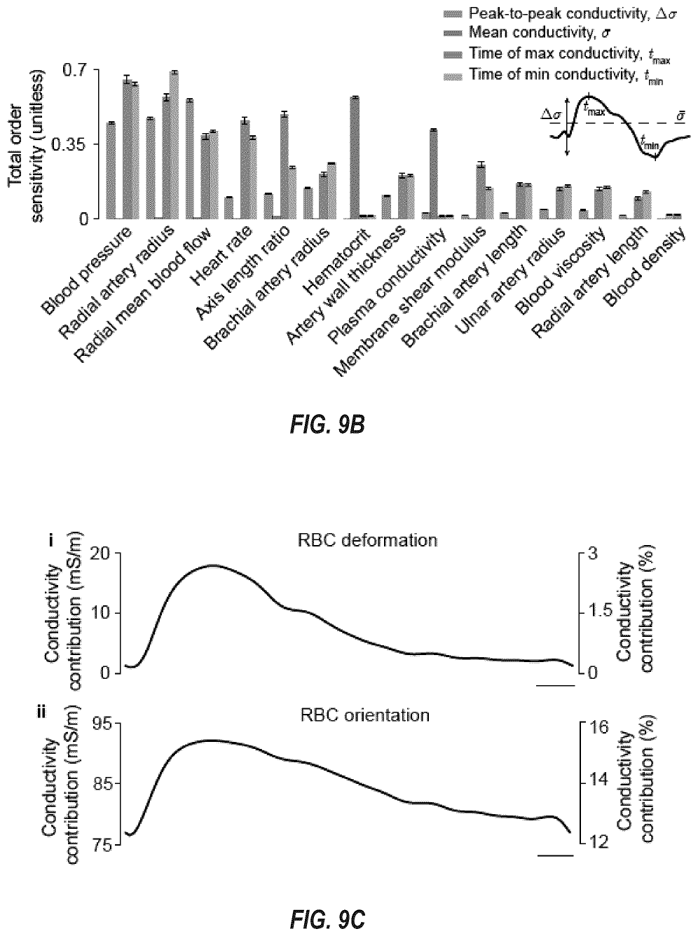

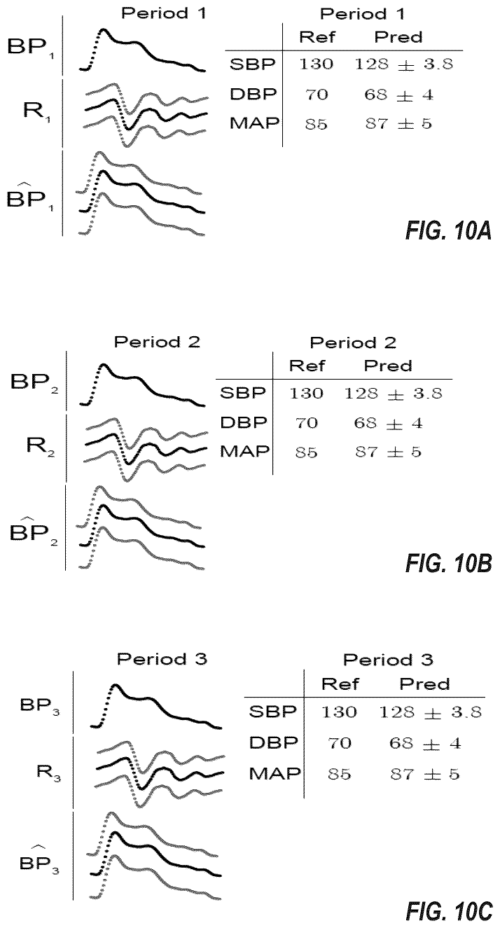

Further, we performed a volume impedance density (VID) analysis at 50 kHz. We calculated the volume within the wrist responsible for 99% of the measured resistance (FIG. 81), resulting in (38±7.2)×(43.5±3.4)×(27.3±5.7) mm³, and quantified the proportional contribution of individual tissues (FIG. 8J). We found that the primary four contributors to the baseline resistance were muscle with 43%+8%, SAT with 26%+10%, skin with 16%+10%, followed by radial artery with 7%+0.008%. This beneficially demonstrates that the radial artery contributes significantly to impedance measured at the skin.

### 2. Coupled Fluid Dynamic and Electrostatic Models

The combined fluid dynamic and electrostatic models (i.e., the full forward model as described herein) was used to relate brachial blood pressure waveforms all the way to impedance measured at the wrist (FIG. 9A). This end-to-end modeling framework allowed us to study how fluid dynamics (brachial BP and mean blood flow), anatomical (radial artery radius), and physiological (hematocrit concentration and plasma conductivity) parameters altered radial BP, blood velocity, shear stress, blood conductivity, and resistance measured at the wrist.

An increase of BP during systole was found to increase blood conductivity and decrease resistance, these temporal features were maintained when considering different brachial mean arterial pressure signals (FIG. 9A, column i). Increasing brachial mean blood flow lowered radial BP, increased both centerline velocity and shear stress, flattened the conductivity with a slower rate of decrease during diastole, and impacted the resistance during diastole with a reduced increase rate (FIG. 9A, column ii). Anatomic variation in the average radius of the radial artery impacted the overall morphology, latency, and duration of fluid and electrical signals (FIG. 9A, column iii). Although physiological variation in hematocrit concentration (FIG. 9A, column iv) and plasma conductivity (FIG. 9A, column v) added an offset to the conductivity and resistance signals, they did not affect the shape of the waveform, unlike blood pressure differences.

Global sensitivity analysis (FIG. 9B) found that blood pressure, radial artery radius, and radial mean blood flow had the largest impact on the peak-to-peak resistance, time of minimum resistance, and time of maximum resistance. This indicates beneficially that blood pressure is a significant contributor to aspects of the impedance signal. The sensitivity analysis was performed as in the sensitivity analysis of the electrostatic model.

To describe impacts of RBC orientation and deformation, for one set of parameters, conductivity can be modeled for stationary blood (no deformation or orientation), for pulsatile flow with only orientation impacts, and finally with both deformation and orientation impacts. The deformation impact is measured by comparing the results of the full model and the model with deformation removed, and the orientation impact is measured by comparing the results of the model with deformation removed and of stationary blood. Those values are plotted, both as a percent and as an absolute change in mS/m, in FIG. 9C. RBC orientation had a larger contribution (12-15%) to the bulk conductivity over a single period than RBC deformation (0.1-3%).

### 3. Accurate Prediction of Synthetic Blood Pressure Using the Predictive Model

We generated a dataset of over 1.2M synchronized experimental BP (from the publicly available PulseDB) and synthetically generated resistance signals (see FIG. 6 and corresponding description). We used this synthetic dataset to train and test the predictive model shown in FIG. 3B. FIGS. 10A-10C show three sample blood pressure waveforms (BPᵢ) with their corresponding resistance (Rᵢ) and predicted blood pressure ( [symbol] ) with uncertainty measures due to anatomical variation. The predictive model was able to generate predicted blood pressure that effectively corresponds to the reference blood pressure.

### Additional Terms & Definitions

The system can be “robust” to certain variations in wearable device placement or user anatomical differences. This means that the system is able to perform without a statistically significant drop in accuracy or effectiveness within the associated range of wearable device placement or user anatomical differences.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “dataset”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, machine learning techniques or algorithms that are not specifically described herein may optionally be specifically omitted from the described embodiments.

1. A system for determining blood pressure from electrical impedance measurements, the system comprising:
a wearable device configured to be worn against or attached to the skin above a target blood vessel, the wearable device comprising an array of electrodes that enable measurement of electrical impedance at the skin;
one or more processors; and
one or more hardware storage devices including instructions stored thereon that are executable by the one or more processors to cause the system to at least:
receive a series of electrical impedance measurements from the wearable device;
based on the series of electrical impedance measurements, calculate a blood pressure prediction using a predictive model that comprises a physics-informed neural network (PINN), the PINN being trained to minimize a first loss function,
wherein the first loss function incorporates the incompressible Navier-Stokes equations for a tube with elastic boundary conditions.

2. The system of claim 1, wherein the predictive model further comprises a decoder neural network configured to output an electrical impedance prediction based on the blood pressure prediction, the decoder neural network being trained to minimize a second loss function that determines loss between measured impedance and the impedance prediction, and the PINN being further trained to minimize the second loss function.

3. The system of claim 2, wherein the decoder neural network is a 2-dimensional convoluted neural network (2D-CNN).

4. The system of claim 2, wherein the decoder neural network is trained using a training dataset that uses a forward model relating blood pressure to electrical impedance measured at the skin.

5. The system of claim 4, wherein the forward model incorporates (1) a physiological model that uses (i) Navier-Stokes equations relating blood pressure to shear stress and red blood cell orientation and (ii) Maxwell-Fricke equations relating red blood cell orientation to blood conductivity, and (2) an electrostatic model relating blood conductivity to impedance measured at the skin.

6. The system of claim 5, wherein the forward model is based on brachial artery blood pressure measurements with blood pressure waveform propagation modeled as branching between radial and ulnar arteries, and wherein the radial artery is the target vessel.

7. The system of claim 5, wherein the electrostatic model includes a muscle tissue layer, a subcutaneous adipose tissue (SAT) layer, and a dermal tissue layer, with a target vessel region embedded within the muscle tissue layer.

8. The system of claim 4, wherein the training dataset incorporates a biological distribution of one or more physiological parameters to relate blood pressure to skin impedance using the forward model.

9. The system of claim 8, wherein the one or more physiological parameters include target artery radius, hematocrit, and/or plasma conductivity.

10. The system of claim 1, wherein the predictive model further comprises a preliminary encoder neural network that functions to reduce dimensionality of a resistance signal before passing a reduced-dimension resistance signal to the PINN.

11. The system of claim 10, wherein the preliminary encoder neural network is a 1-dimensional convoluted neural network (1D-CNN).

12. The system of claim 1, wherein the wearable device comprises a wireless communications module enabling communication with one or more external devices, and wherein the one or more external devices receive the series of electrical impedance measurements and calculates the blood pressure prediction therefrom.

13. The system of claim 1, wherein the wearable device is configured to be worn on the wrist.

14. The system of claim 13, wherein the target blood vessel is the radial artery.

15. The system of claim 1, wherein the wearable device is configured in a patch form factor.

16. The system of claim 1, wherein the calculated blood pressure prediction comprises a blood pressure waveform.

17. The system of claim 1, wherein the system is robust to positioning of the wearable device within at least 10 mm to either side of the target blood vessel.

18. The system of claim 1, wherein the system is robust to differing target artery depths within at least ±2 mm from a median target blood vessel depth.

19. The system of claim 1, wherein the blood pressure prediction is calculated in real time.

20. A computer-implemented method for determining blood pressure from electrical impedance measurements received from a wearable device configured to be worn against or attached to the skin above a target blood vessel, the method comprising:
receiving a series of electrical impedance measurements from the wearable device;
based on the series of impedance measurements, calculating a blood pressure prediction using a predictive model that comprises
(1) a physics-informed neural network (PINN), the PINN being trained to minimize a first loss function, wherein the first loss function incorporates the incompressible Navier-Stokes equations for a tube with elastic boundary conditions, and
(2) a decoder neural network configured to output an impedance prediction based on the blood pressure prediction, the decoder neural network being trained to minimize a second loss function that determines loss between measured impedance and the impedance prediction, and the PINN network being further trained to minimize the second loss function.
